(19) United States
(12) Patent Application Publication
(10) Pub. No.: US 2024/0416350 A1
(43) Pub. Date: Dec. 19, 2024

(54) GLASS ELEMENT AND SYSTEM FOR INVESTIGATING BIOLOGICAL MATERIAL, AND PROCESS FOR PRODUCING SAME

(71) Applicant: Schott AG, Mainz (DE)

(72) Inventors: Johanna Blass, Mainz (DE); Stephanie Mangold, Mainz (DE); Oliver Sohr, Mainz (DE)

(73) Assignee: Schott AG, Mainz (DE)

(21) Appl. No.: 18/743,473

(22) Filed: Jun. 14, 2024

(30) Foreign Application Priority Data
Jun. 15, 2023 (DE) 10 2023 115 623.3

(51) Int. Cl. B01L 3/00

(52) U.S. Cl. CPC B01L 3/5085; B01L 2300/0861

(57) ABSTRACT

A glass element for receiving and/or conveying biological material includes a multiplicity of micro-channels, the micro-channels tapering from a bottom side of the glass element in a direction of a top side of the glass element.

### CROSS REFERENCE TO RELATED APPLICATIONS

This application claims priority to German Patent Application No. 10 2023 115 623.3 filed on Jun. 15, 2023, which is incorporated in its entirety herein by reference.

### BACKGROUND OF THE INVENTION

### 1. Field of the Invention

The invention relates to a glass element having micro-channels that can be used in particular for the spatially resolved detection and investigation of biological material. Examples of such biological material are human, animal or plant proteins, antibodies and/or DNA of a cell which is part of a cell assembly, such as a tissue sample or cell culture, for example. The invention likewise relates to a system for investigating biological material and to a process for producing the glass element. The investigations referred to above are generally called cell and/or DNA diagnostics. The glass element of the invention allows the biological material to be investigated with spatial resolution, also referred to as “spatial diagnostics” or “spatial biology”. The invention referred to above allows the spatially resolved investigation of expression patterns of DNA and proteins of a cell, with retention of the information relating to the cell assembly, such as a tissue sample or cell culture, for example. It also allows single molecule investigations, of biomarkers in biological or medical samples, for example, such as, in particular, blood, serum or urine samples, cell supernatants, etc.

### 2. Description of the Related Art

Glass elements for the separation of DNA in liquid media are known from US 20150122656 A1. In that application, channels are drilled into a glass plate, using laser beams. The process described is based on the introduction of filaments into the glass by means of consecutive pulses of an ultrashort pulse laser. By shifting the focus of the laser, channels passing through the substrate are formed from the filaments, with a diameter of 1 μm. It is reported that particularly smooth inner walls of the channels generated are achieved with this process, and deviate negligibly, if at all, from the ideal cylinder shape.

For spatially resolved investigations, it is desirable to arrange as many channels as possible as close as possible to one another in the glass element. It is also desirable to tailor the diameter of the channels so as to achieve sufficient collection efficiency for the biological material. However, every channel represents a weakening of the glass element, and there must be a sufficient glass volume between the channels to achieve a sufficient minimum stability that ensures that the glass element can be rationally used. There is therefore a conflict of objectives between collection efficiency and spatial resolution.

### SUMMARY OF THE INVENTION

In some embodiments provided according to the present invention, a glass element for receiving and/or conveying biological material includes a multiplicity of micro-channels. The micro-channels taper from a bottom side of the glass element in a direction of a top side of the glass element.

In some embodiments provided according to the present invention, a system for investigating biological material includes: a glass element having a multiplicity of micro-channels, the micro-channels tapering from a bottom side of the glass element in a direction of a top side of the glass element, the glass element in an operating state serves for receiving and/or conveying the biological material in the or through the micro-channels from the bottom side of the glass element into a region of the top side of the glass element; and an evaluation facility with which in the operating state the biological material can be investigated.

In some embodiments provided according to the present invention, a process for producing a glass element having a plurality of micro-channels which connect one surface of the glass element to an opposite surface of the glass element is provided. The micro-channels taper from a bottom side of the glass element in a direction of a top side of the glass element. The process includes: providing an ultrashort pulse laser; providing a glass element base body; directing a pulsed laser beam of the ultrashort pulse laser onto the glass element base body; harmonizing a wavelength of the laser beam and a material of the glass element base body with one another such that the glass element base body is substantially transparent to the laser beam; focusing the laser beam to a focus region which is elongated in a beam direction and which lies at least partly within the glass element base body, an intensity of the laser beam and an extent of the focus being of a magnitude such that the laser beam leaves a filamentary flaw in the glass element base body, the focusing inserting a plurality of such filamentary flaws; and etching open the filamentary flaws to give micro-channels.

### BRIEF DESCRIPTION OF THE DRAWINGS

The above-mentioned and other features and advantages of this invention, and the manner of attaining them, will become more apparent and the invention will be better understood by reference to the following description of embodiments of the invention taken in conjunction with the accompanying drawings, wherein:

FIGS. 1A to 1D is illustrate different embodiments of glass elements with widened micro-channels, in section;

FIG. 2 illustrates a section through a detail of a glass element with widened micro-channels;

FIG. 3 illustrates an electron micrograph of a wall of a micro-channel;

FIG. 4A and FIG. 4B illustrate the plan view of the top and bottom sides of glass elements;

FIG. 5 illustrates a micrograph of a section through a glass element;

FIG. 6 illustrates a system for the spatially resolved investigation of biological material;

FIG. 7 illustrates the coverage of dyes on the top side of a glass element;

FIG. 8 illustrates the coverage of dyes in blind holes of a glass element;

FIG. 9 illustrates an apparatus for the laser treatment of the glass elements in preparation for subsequent etching;

FIG. 10A illustrates schematically the effect of the structure of a smooth micro-channel wall; and

FIG. 10B illustrates schematically the effect of the structure of a structured micro-channel wall.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION OF THE INVENTION

The invention encompasses a glass element for receiving and/or conveying biological material 60, more particularly human or animal or plant cells and/or cell constituents, more particularly proteins and/or DNA and/or RNA, having a multiplicity of micro-channels which optionally connect one surface of the glass element to the opposite surface of the glass element or end in a blind hole, wherein micro-channels taper from the bottom side in the direction of the top side. Optionally, micro-channels are at least regionally funnel-shaped.

The bottom side of the glass element is the side which in use is facing the biological material for investigation. The glass element is typically a platelike element, referred to as a slide. The micro-channels may connect one surface of the glass element to the opposite surface of the glass element, more particularly the bottom side to the top side. In that case, the micro-channels are continuous. Alternatively, however, it is also possible for the micro-channels to have a blind hole embodiment, in which case they do not penetrate one side, by definition the top side. The base of the blind hole in that case is located in the region and/or vicinity of the surface. The micro-channels widen from the side of one surface of the glass element in the direction of the opposite surface, and optionally are at least regionally funnel-shaped.

In some exemplary glass elements, correspondingly, the diameter of the channel entrance of a micro-channel at the bottom side is greater than the diameter of the channel exit or channel end in the region of the top side.

This may be viewed as a consequence of the widening of the micro-channels. On one side of the glass element, the channels have an opening whose diameter is greater than the diameter on the opening or of the end of the blind hole on the opposite side. The surfaces are typically the main sides of a platelike glass element. The bottom side thereof is defined as the side which in use is facing the biological material for investigation, and the top side is the opposite side, which is facing the investigation facility. The widening of the channel is typically located on the bottom side and is therefore facing the biological material for investigation. It may be advantageous for the micro-channels to have a circular or oval diameter. Micro-channels in the sense of the present invention mean that the diameter thereof is in the range from a few micrometers up to a few hundred micrometers.

The widening of micro-channels at the bottom side of the glass element and hence the fundamental funnel shape thereof increases the collection efficiency for the biological material, while there is more glass material between the micro-channels on the top side of the glass element, so ensuring the mechanical stability of the glass element. Accordingly, the openings of the channels on the bottom side of the glass element can be arranged very close to one another and hence a good spatial resolution can be achieved, while still providing good mechanical stability.

Also possible and part of the invention is a double funnel, in which the widenings of micro-channels are present on the top side and on the bottom side of the glass element. In the middle region of the micro-channels, the channels are, so to speak, constricted and/or cylindrical and there is sufficient glass material between these regions and hence in the volume of the glass element.

Some embodiments relate to a glass element wherein the micro-channels at least in the region of a channel entrance or channel exit in the region of a surface of the glass element have a diameter of 5 μm to 200 μm, optionally of 7 μm to 130 μm, optionally of 10 μm to 100 μm.

As a result of the choice of these diameters, biological material is able to penetrate into micro-channels on one side of the glass element, typically the bottom side as described, and be transported in the direction of the opposite surface. The selection of the diameters makes a contribution to, or determines, the ability only of the biological material for investigation, characteristically with lengths of less than 1 μm, to enter the micro-channels, but not unwanted larger cells and/or cell constituents.

In some exemplary glass elements, the channel entrance has a diameter of 5 μm to 200 μm, optionally of 10 μm to 100 μm.

As described, this may be a conical region which tapers in the direction of the opposite surface, or the widened region may be adjoined by a cylindrical region of largely constant diameter, or a region which widens in the direction of the opposite surface. It is also possible for the cylindrical region to be adjoined by a widening region.

The opening angle of the funnel-shaped region of all the micro-channels described is optionally from 0.10 to 30°, optionally from 2° to 18°. The thickness of the glass element is optionally 0.1 mm to 3 mm.

The selection of the specified opening angles enables efficient production, more particularly by the processes described later on below. These values also contribute to the ability of the biological material for investigation to be transported effectively from the channel entrance in the direction of the other surface without any hold-up.

The selection of the opening angle allows concentration of the material for investigation within a channel and/or separation between two or more channels of the biological material at the top side of the glass element. The concentration is proportional to the ratio of the square of the channel diameters (R_{bottom})²/(Rₜₒₚ)², also called radii ratio, and increases the sensitivity of the analysis by fluorescence spectroscopy and/or enables targeted interaction with functional molecules at the channel wall. Concentration may be particularly advantageous at a radii ratio of more than 5.

The process of the invention, which is described below, enables production in this broad range, but advantageously, in particular, for the very low thicknesses.

In accordance with the observations above, in some exemplary glass elements, the funnel-shaped region in the region of the channel entrance is adjoined by a cylindrical or funnel-shaped region, more particularly in the region of the channel exit and/or the end of the blind hole.

As a result of the stated features, it is possible for the glass element to be able to have a very high density of micro-channels. The density of micro-channels is also called micro-channel density and indicates the number of micro-channels per unit area on the surface of the glass element—for example, the number of micro-channels per square millimeter. Since the channel entrance is always assigned to a channel exit or end of a blind hole, the micro-channel density on the top side of the glass element is equal to the micro-channel density on the bottom side of the glass element. It may therefore be called a general micro-channel density.

With particular advantage, a glass element provided in accordance with the invention has a micro-channel density of at least 20/mm² or at least 50/mm², more particularly at least 400/mm², more particularly at least 10 000/mm² as a lower limit. The upper limit may in each case advantageously be at most 16 000/mm².

In some embodiments of the glass element, the channel wall comprises a structure having a multiplicity of rounded, hemispherical recesses, which in particular have a depth of less than 5 μm and more particularly have an extent of, in particular, 5 the 20 μm; optionally, the roughness is from 50 nm to 1 μm. The value relates to the roughness average Ra.

The inventors have recognised that it is advantageous, surprisingly, for the wall of the micro-channels not to be as smooth as possible, but instead to have a structure. The stated depth and roughness represent optional embodiments. Depth here means the recession in a direction perpendicular to the axis of the channel; extent means the length of the recesses parallel to the axis of the channel.

These structures can be rationally produced by the ultrashort pulse laser production process with subsequent etching step, described later on below.

An advantage provided by these hemispherical recesses is that when gel is introduced into the micro-channels, it not only can be introduced well but also likewise remains adhering well in the channels.

A further particular advantage of the structure of the channel wall, more particularly of the hemispherical recesses, is the possibility for adjustment of the electro-osmotic flow (EOF). The electro-osmotic flow is a charge-induced movement of liquids along a polar surface in an electrical field—for example, a buffer solution in a glass capillary.

The selection of the roughness permits the possibility for adjustment of the contribution of the EOF to the movement of the biological material in the channels. A greater roughness of the polar surface leads to a reduction in the velocity of the EOF and possible reversal of the direction of movement of the polar biological material in the glass element, in comparison to a surface with lower roughness.

The selection of the channel wall roughness with simultaneously adjustable opening angle permits the selective closing of the channel by opposite EOF and applied electrical field.

In some exemplary glass elements, the channel exits form an exit matrix in which the position of the channel entrance is assignable to each channel exit.

By this in particular it is possible to perform spatially resolved investigation of biological material. This in particular because the channel exits or, alternatively, the positions of the blind holes form an exit matrix in which the position of the channel entrance is assignable to each channel exit.

This means, in other words, that the position of the entrance matrix can be assigned to the position of the exit matrix. At its most simple, the center point of a channel entrance on the bottom side of the glass element coincides at least substantially with the position of the channel exit on the top side of the glass element or on the end of the blind hole. This corresponds to a version of the micro-channels in which the axes of channels run perpendicularly through the main faces of the glass element. It is also possible, however, for the channel axes to run diagonally through the main faces of the glass element. In this case as well, however, the position of the channel entrance can be assigned to the position of the channel exit or of the end of the blind hole, so that analysis of the biological material for investigation at the channel exit or at the end of the blind hole is an unambiguous indication of its position in relation to the entrance matrix and/or the channel entrance.

The glass element, at least in the region of the micro-channels, optionally comprises a glass which is notable for having a low intrinsic fluorescence in the wavelength range from 300 nm to 700 nm.

The intrinsic fluorescence ought to be less than 10⁻⁶ of the irradiated light intensity. Because of the low intrinsic fluorescence, the fluorescent light does not interfere with the optical investigation of the biological material. Suitable intrinsic fluorescence ranges are, for example, from 10⁻⁸ to 10⁻⁶ of the irradiated light intensity.

Particularly suitable glass compositions are given later on below in the examples. Particular suitability is possessed in general by glasses distinguished by high chemical stability and/or high water resistance. Alkali-free glasses may be especially advantageous. Suitable glass materials may in particular be alkali-free glasses and borosilicate glass. Particular candidates are commercially available glasses having the product designations AF32, AF35, AS87, D263, D263T, B270, MEMPAX, Willow, G-Leaf, EN-A1 and BDA-E.

According to some embodiments, the composition of the glass comprises constituents as follows in percent by weight:

| Composition | (% by weight) |
|---|---|
| SiO₂ | 63-85 |
| Al₂O₃ | 0-10 |
| B₂O₃ | 5-20 |
| Li₂O + Na₂O + K₂O | 2-14 |
| MgO + CaO + SrO + BaO + ZnO | 0-12 |
| TiO₂ + ZrO₂ | 0-5 |
| P₂O₅ | 0-2. |

According to some embodiments, the composition of the glass comprises constituents as follows:

| Composition | (% by weight) |
|---|---|
| SiO2 | 60-84 |
| Al₂O₃ | 0-10 |
| B₂O₃ | 3-18 |
| Li₂O + Na₂O + K₂O | 5-20 |
| MgO + CaO + SrO + BaO + ZnO | 0-15 |
| TiO₂ + ZrO₂ | 0-4 |
| P₂O₅ | 0-2. |

According to some embodiments, the composition of the glass comprises constituents as follows:

| Composition | (% by weight) |
|---|---|
| SiO₂ | 58-65 |
| Al₂O₃ | 14-25 |
| B₂O₃ | 6-10.5 |
| MgO + CaO + SrO + BaO + ZnO | 8-18 |
| ZnO | 0-2. |

According to some embodiments, a further suitable composition of the glass is given by:

| Composition | (% by weight) |
|---|---|
| SiO₂ | 50-81 |
| Al₂O₃ | 0-5 |
| B₂O₃ | 0-5 |
| Li₂O + Na₂O + K₂O | 5-28 |
| MgO + CaO + SrO + BaO + ZnO | 5-25 |
| TiO₂ + ZrO₂ | 0-6 |
| P₂O₅ | 0-2. |

According to some embodiments, the composition of the glass comprises constituents as follows:

| | |
|---|---|
| SiO₂ | 52-66 |
| B₂O₃ | 0-8 |
| Al₂O₃ | 15-25 |
| MgO + CaO + SrO + BaO + ZnO | 0-6 |
| ZrO₂ | 0-2.5 |
| Li2O + Na2O + K2O | 4-30 |
| TiO₂ + CeO₂ | 0-2.5. |

For all of the glass compositions referred to above, coloring oxides may optionally be added, such as Nd₂O₃, Fe₂O₃, CoO, NiO, V₂O₅, MnO₂, CuO, Cr₂O₃.

0-2% by weight of As₂O₃, Sb₂O₃, SnO₂, SO₃, Cl, F and/or CeO₂ may be added as refining agents, and the total amount of the overall composition is in each case 100% by weight.

It may also be advantageous, and is encompassed by the invention, for the surface energy of the walls of micro-channels to be adjusted such that the biological material for investigation is conveyed by the walls or the biological material for investigation adheres to the walls. The surface energy may be adjusted, for example, by coatings.

In some exemplary glass elements, therefore, at least walls of the micro-channels are coated, more particularly with coatings which repel the biological material for investigation or to which the biological material for investigation adheres. In particular for optimizing the surface energy, the coating optionally comprises, at least regionally, polymers, silanes, oligonucleotides, antibodies, proteins, antigens or nitrocellulose or inorganic coatings, more particularly metallic particles, especially gold and/or iron, or polymer-based nanoparticles or microparticles, or combinations of those stated.

As described before, it is possible for the coating to be present only in portions of the micro-channels, for example at the channel entrance, or for different coatings to be present in the channel, in particular differently along the channel axis.

The coating may typically have thicknesses of one molecular monolayer or coatings with thicknesses in the nm range up to the complete filling of the channel. In the simplest case, the coating is applied via dip coating processes. Particularly in the case of low channel diameters, coating takes place suitably by vacuum processes, e.g. chemical vapor deposition (CVD) or atomic layer deposition (ALD). It may be advantageous to attach the coating selectively in the channels, by means, for example, of a lift-off material outside the channels or selective sputtering onto the surface of the glass element.

The stated advantageous structure of the walls of the micro-channels works together, with particular advantage, with the coatings and/or fillings, particularly since the structure increases the surface area of the channel walls. In the case of adhering coatings, there is then more area for biological material for investigation; in the case of conveying coatings, the coating suppresses or prevents the clogging of the structures with biological material and/or at least contributes thereto. In the case of fillings, the adhesion of the filling in the channels can be assured or at least improved.

In some glass elements, the micro-channels are filled with a gel. The gel optionally has pores. Depending on the choice and/or configuration of the gel, the pores can have different diameters, allowing them to receive and/or convey biological material of different sizes.

Especially suitable for the purposes of the invention are polyacrylamide gel and/or agarose gel. Agarose gel typically has larger pores than polyacrylamide gel and can be used in particular for the analysis of DNA and/or larger proteins. Polyacrylamide gel, with its generally smaller pores, is used typically for the analysis of smaller proteins.

The gel, more particularly the stated gels, may be used for the purposes of the invention as carrier gel in an electrophoresis process, typically also called gel electrophoresis. This process can be used in particular for separating various small molecules, especially DNA, RNA and proteins, from one another and analysing them in subsequent steps. In the present case, the concept of gel electrophoresis generally embraces the conveying and/or moving of the substances for investigation through the gel; separation is not absolutely necessary.

Here, the molecules for separation and/or conveying move through the carrier gel, which is located optionally in the micro-channels. The movement may be assisted, as further described, by the application of an electrical field. The molecules migrate to different extents and/or at different rates according to the magnitude and charge and/or strength of the electromagnetic field applied.

With conventional electrophoresis, the molecules form a characteristic band pattern within the micro-channel. The molecules can be visualised by staining; the characteristic colouration of a molecule can be assigned to the molecule and so the molecule can be identified.

In the case of the glass elements provided according to the invention, especially with thin glass elements, however, it is also envisaged that the gel and/or alternative substances serve for the electrophilic conveying of the material for investigation from the bottom side of the glass element to the top side of the glass element. The analysis then takes place on the top side, by fluorescence spectroscopy or through DNA sequencing by nanopores, for example. These are standard processes and are not set out further in detail here.

In some glass elements, at least one DNA- or RNA-sensitive dye, more particularly a plurality of dyes, is or are located in the region of the channel exits or at the base of the blind holes.

When DNA and/or RNA is transported from the biological material for investigation through the micro-channels, it reaches the channel exit and/or the base of the blind hole, if the micro-channels are embodied as such. There, DNA or RNA can react with a DNA- or RNA-sensitive dye attached there. The characteristic fluorescence, more particularly its wavelength, then verifies the presence of the corresponding DNA or RNA strand.

Examples of possible advantageous dyes may be ethidium bromide, propidium iodide, crystal violet, 4′,6-diamidino-2-phenylindole and/or 7-aminoactinomycin D.

In some exemplary configurations of the glass element, molecules for the polymerase chain reaction, more particularly DNA, primers, nucleotides and/or the enzyme DNA polymerase, are located in the region of the channel exits or at the base of the blind holes. Such embodiments may be particularly suitable for single molecule investigation, in which, as described herein, the material for investigation is first multiplied in the micro-channels and/or blind holes. It is apparent that the aforesaid molecules may in particular also be introduced into the micro-channels and/or blind holes when the material for investigation is introduced into these same micro-channels/blind holes.

A possible advantage of such embodiments is that the cells and/or DNA and/or RNA in the material for investigation are multiplied in the micro-channels (including blind holes) in a very small volume. As a result, the time needed for the required multiplication can be reduced. Similarly, investigation, including verification, of the material for investigation can take place in this small volume, resulting in high capacity for evaluation, more particularly good signal quality. Because of the enhanced accuracy, in particular, scarce populations can be identified or subtle differences between cells can be recognized. New biomarkers or signature molecules can be identified accordingly. Moreover, with an investigation of the individual molecules/cells in the sample, a conclusion can be drawn as to the heterogeneity of the molecules/cells in the sample. In particular, individual differences between patients can be visualised for the development of personalized treatment approaches, based on the specific properties of the cells/molecules. All in all, therefore, a rational and reliable investigation is provided which has potential applications in personalized medicine.

The glass element described is, so to speak, the precursor product of the system provided according to the invention with which spatially resolved diagnosis and/or single molecule diagnosis or single cell diagnosis can be performed. The biological material may, as described, be analyzed and/or multiplied in an ancillary process on the glass element or by DNA sequencing, for example. Hence it is possible at the same time to determine the expression pattern of genes and/or proteins and the distribution pattern of vDNA markers of individual cells, with the spatial information in the cell assembly or tissue sample being retained.

The system for investigating biological material, more particularly human or animal or plant cells and/or cell constituents, more particularly proteins and/or DNA and/or RNA, comprises a glass element having a multiplicity of micro-channels, more particularly an above-described glass element, wherein the glass element in the operating state serves for receiving and/or conveying the biological material in the or through the micro-channels from the bottom side of the glass element into the region of the top side of the glass element, having an evaluation facility with which in the operating state the biological material can be investigated. For the purposes of the invention, the evaluation facility is assigned or subordinate to the glass element.

The evaluation facility generally captures the presence of biological material and/or constituents thereof transported through and/or present in the micro-channels. In particular, the evaluation facility may comprise an electronic image sensor, capable more particularly of recording the intrinsic fluorescence of the aforesaid dyes. The configuration of the micro-channels as blind holes is therefore also encompassed.

In some exemplary system, therefore, at least one DNA- or RNA-sensitive dye is located in the region of the channel exit 22 and/or in the blind hole 25, more particularly at the base thereof, the evaluation facility being able to capture, more particularly to capture with spatial resolution, the colour information of said dye.

In the case of DNA investigations, a typical DNA-binding dye is, for example, ethidium bromide.

The system may advantageously comprise a sample carrier, on which the biological material for investigation is attached in the operating state. This carrier may typically be a small sample plate or a slide.

In some embodiments of the system, a transport facility is assigned to the glass element and in the operating state introduces the biological material into the micro-channels of the glass element.

The transport facility is generally a facility which exerts a force on the biological material for investigation that at least assists the movement and/or transport of said material into the micro-channels of the glass element and in the direction of its opposite surface.

In some exemplary systems, the transport facility comprises a negative pressure unit with which, in the operating state, negative pressure is applied to the exit side of the glass element, and/or a facility for generating a potential gradient over the thickness of the glass element, so that biological material for investigation is introduced into and/or transported through the micro-channels by a flow of ions or by an electrical field.

Because the phosphate radicals of DNA give it a fundamentally negative charge, it migrates to the anode in the gel electrophoresis. With particular advantage, therefore, an anode is attached on the top side of an exemplary glass element, or the top side is assigned to an anode. When a cathode is located on the bottom side or in the vicinity of the bottom side, the application of voltage produces an electrical field and the anions migrate to the anode and the cations migrate to the cathode.

In the case of DNA investigation, it is of course possible to multiply the DNA portions beforehand and/or in the channels of the glass element, more particularly by polymerase chain reaction.

Depending on the biological material for investigation, it is of course also possible, and encompassed by the invention, for the anode to be provided on the bottom side of the glass element and the cathode on the top side, and/or to be assigned thereto in each case. Other arrangements are also possible and encompassed by the invention. For example, it may be sufficient for the medium in which the biological material for investigation is located to be set in motion by the transport facility.

The transport facility may comprise a squeegee unit, with which biological material for investigation can be introduced into the micro-channels, more particularly the blind holes, by sweeping.

Some exemplary systems comprise as transport facility a microfluidic system which introduces the biological material for investigation into the blind holes and/or micro-channels by fluid flow.

With particular advantage, the system may be developed such that the evaluation facility comprises a closed or nanoporous membrane composed more particularly of a non-conductor or a semiconductor or the combination of non-conducting, conducting and/or semiconducting constituents, more particularly glass, silicon, graphene, and/or biomolecules, more particularly lipid molecules, DNA origami structures or transmembrane proteins, on the exit side of the micro-channels, with which the biological material can be investigated for, in particular, the sequence or the binding behaviour or the size or the optical properties, more particularly the fluorescence.

In some embodiments, the system comprises an evaluation facility in the form of a closed or nanoporous membrane, composed more particularly of a non-conductor or a semiconductor or the combination of non-conducting, conducting and/or semiconducting constituents, more particularly glass, silicon, graphene, and/or biomolecules, more particularly lipid molecules or transmembrane proteins, on the top side of the micro-channels. With this, the biological material for investigation is investigated for, in particular, the sequence or the binding behaviour or the size or the optical properties, more particularly the fluorescence.

As described, the micro-channels may be filled with a gel to assist transport in particular. The gel itself may contain a dye which binds to the biological material for investigation. In the case of DNA investigations, this is a DNA-binding dye such as ethidium bromide, for example. It is also possible for the sample to be located in a sample buffer which in turn contains a dye, such as crystal violet, for example. Alternatively, after the electrophoresis, the gel may be stained with a DNA-binding dye, for example with methylene blue, Stains-all or ethidium bromide. In the case of fluorescent dyes such as ethidium bromide, it is often necessary to illuminate the agarose gel with UV light and a UV filter on the photographic camera. RNA investigations are of course also possible.

In some exemplary systems, biological material is multiplied in micro-channels and/or blind holes in the operating state, often called digital PCR. Molecules for the polymerase chain reaction are present in particular in the region of the channel exits or at the base of the blind holes.

If the blind holes contain molecules for the polymerase chain reaction (PCR), DNA or RNA molecules present in the material for investigation can be multiplied. They include, for example, DNA, primers, nucleotides and the enzyme DNA polymerase.

Because of the temperature-stable properties of the glass element, possibilities include not only room temperature PCR, which is typically performed at up to 40° C., but also standard PCR at up to almost 100° C. At these temperatures, no adverse effect on the spatial information as a result of the thermal expansion or shifting of the position of the blind holes relative to one another can be expected, in contrast to plastics. Furthermore, the good thermal conductivity of glass by comparison with plastics has the advantage that the heat is distributed uniformly in the glass element and homogeneous PCR throughout the sample can be ensured. Furthermore, by comparison with plastic, glass has the advantage of little intrinsic fluorescence, and so a high signal strength is achieved in comparison to the background.

Since, as described, the exit matrix of the micro-channels on the top side of the glass element may be assigned to the entrance matrix of the micro-channels on the bottom side of the glass element, the system described is capable of concluding the position of the entrance of the corresponding biological material on the bottom side from the position of an investigation result on the top side. It is possible, so to speak, to assign a location of the origin of the biological material in the sample to the location of a micro-channel on the glass element. Accordingly, the glass element provided according to the invention and/or the system provided according to the invention are/is capable of permitting spatially resolved investigation of samples of biological material. This is also called “spatial biology” or “spatial diagnostics”.

The glass element described may be produced rationally and in large quantities by the process provided according to the invention, which is elucidated below. The process comprises the process steps of
providing an ultrashort pulse laser,
providing a glass element base body,
a pulsed laser beam of the ultrashort pulse laser is directed onto the glass element base body,
the wavelength of the laser beam and the material of the glass element base body being harmonized with one another such that the workpiece is substantially transparent to the laser beam, and
the laser beam being focused to a focus region which is elongated in beam direction and which lies at least partly within the glass element base body,
the intensity of the laser beam and the extent of the focus being of a magnitude such that the laser beam leaves a filamentary flaw in the workpiece, and a multiplicity of such filamentary flaws being inserted,
etching open the filamentary flaws to give micro-channels.

The glass element base body may in general be a small glass plate with the desired dimensions. A pulsed laser beam of the ultrashort pulse laser is directed onto the glass element base body, with the wavelength of the laser beam and the material of the glass element base body being harmonized with one another such that the workpiece is substantially transparent to the laser beam.

The laser beam is focused to a focus region which is elongated in the beam direction and which lies at least partly within the glass element base body. This may be done using various optical set-ups, for example Bessel lens systems with the use of an axicon, lens systems with chromatic aberration or lens systems with spherical aberration.

The intensity of the laser beam and the extent of the focus are adjusted to a magnitude such that the laser beam leaves a filamentary flaw in the workpiece, with a multiplicity of such filamentary flaws being inserted. The filamentary flaw represents, so to speak, a threadlike flaw in the glass, which is etched open to give micro-channels in a subsequent process step.

The filamentary flaw comes about through a nonlinear optical effect with self-focusing of the laser beam. This requires ultrashort laser pulses in the ps range.

In some exemplary processes, a sequence of laser pulses is used to generate a filamentary flaw.

In this so-called burst operating mode, the laser energy is delivered not as a single pulse but rather as a sequence of pulses emitted closely one after another, together forming a pulsed packet, called a burst. A pulsed packet of this kind typically has a somewhat greater energy than a single pulse in the customary single-shot operation. The pulses of a burst themselves, however, in return contain significantly less energy than a single pulse. For the pulses within one burst, the pulse energies are flexibly adjustable, more particularly such that the pulse energies remain substantially constant or else the pulse energies increase or else the pulse energies decrease.

A suitable laser source may be, for example, a neodymium-doped yttrium aluminium garnet laser having a wavelength of 1064 nanometers. The laser source generates, for example, a raw beam having a (1/e2) diameter of 12 mm; the optical system used may be a biconvex lens having a focal distance of 16 mm. The raw beam may optionally be generated using a suitable beam-shaping lens system, such as a Galilean telescope, for example.

The laser source may in particular operate with a repetition rate of between 1 kHz and 1000 kHz, optionally between 2 kHz and 100 kHz, optionally between 3 kHz and 200 kHz.

The repetition rate and/or the scan rate may be selected such as to achieve the desired distance between adjacent filamentary flaws.

The suitable pulse duration of a laser pulse may be situated in particular in a range of less than 100 picoseconds, optionally at less than 20 picoseconds.

The typical power of the laser source here may be situated in a range from 20 to 300 watts. The laser energy is substantially below the ablation energy of the substrate. To obtain the filamentary flaws, in accordance with some developments provided according to the invention, a pulse energy in the burst of more than 400 microjoules is used, for example a total burst energy of more than 500 microjoules.

When the ultrashort pulse laser is operated in burst mode, the repetition rate is the rate of repetition of the delivery of bursts. The pulse duration is substantially independent of whether a laser is operated in single pulse operation or in burst mode. The pulses within a burst typically have a similar pulse length to a pulse in single pulse operation. The burst frequency may be in the range from 15 MHz to 90 MHz, optionally in the range from 20 MHz to 85 MHz, and for example is 50 MHz, and the number of pulses in the burst may be between 1 and 10 pulses, e.g. 6 pulses.

In some exemplary processes, the elongated focus region comprises a region with maximum energy and a subsequent widened fraction, the elongated focus region being situated such that the widened fraction lies in a surface region of the glass element base body.

The tapering micro-channels may be generated particularly advantageously in this way. The preliminary filamentary flaw is generated, so to speak, with a flaw region which in the subsequent etching step results in the widening at the channel entrance.

In some embodiments of the process, the filamentary flaws do not reach at least one surface of the glass element base body and the opening to this surface is accomplished by the subsequent etching step.

This may likewise be achieved through the choice of the focal position and hence of the intensity of the laser beam. It is thought that, since the generation of the filamentary flaw is a nonlinear effect, a local beam intensity must be attained in order actually to generate the flaw in the glass. This means that, if this critical threshold is not reached, there is de facto no flaw or at least no sufficient flaw in the glass to form a filament. Only the subsequent etching process widens the filamentary flaws and generates, so to speak, the passage through the relevant surface of the glass element base body. The inventors have recognized that the matrix is of the channel exits of the micro-channels can be produced with particular accuracy as a result. Particular accuracy in this sense means that the variance in the diameters of the channel exits is small and/or there is at most a slight deviation from the ideal circular diameter of a channel exit.

The center point of a channel entrance may lie in the simplest case above the center point of a channel exit. In that case, the micro-channel passes, so to speak, perpendicularly through the glass element. However, it is also possible for the center points of channel entrances and channel exits to be offset from one another, so that the micro-channel passes through the glass element at an angle which deviates from the orthogonal through the glass element face. Exemplary suitable angles are those of up to about 10° from this orthogonal, i.e. angles in a range from 0.1° to 10°. It is possible in particular for the passage angles of the micro-channels not to be the same throughout the glass element. The local adjustment of the passage angles allows the channel matrix of the glass element to be adapted to the requirements.

The position of the widening of the channel opening is governed substantially by the focal position. As described, the subsequent etching process widens the flaw in the glass material. The greater the flaw region, the greater the widening.

The properties of the widening of the channel entrance are adjusted by utilizing the shape of the elongated focus. This focus is in fact configured such that a region with maximum energy is adjoined, viewed along the beam axis, by a widened region. The elongated focus region is positioned such that the widened fraction lies in a surface region of the glass element base body.

During the subsequent etching, this becomes the widened channel entrance, more particularly with funnel-shaped widening. The etching step may be performed such that the micro-channels at least in the region of the channel entrance have a diameter of 5 μm to 200 μm, optionally of 7 μm to 130 μm, optionally of 10 μm to 100 μm.

In some embodiments of the process, filamentary flaws do not reach at least one surface of the glass element base body and the opening to this surface is accomplished by the subsequent etching step.

The reason for this is that it is of great interest to provide an extremely accurate geometry of the channel exit as well, which may lie in particular in a cylindrical region of the micro-channel. To adjust the geometry of the channel exit, it has emerged that the filamentary flaw ought advantageously not to pass through the surface of the glass element on which the channel exit is located. The filamentary flaw then ends, so to speak, beneath this surface. This as well is adjusted through the corresponding focal position. In this case, the opening of the micro-channel, i.e. the channel exit, is generated by the subsequent etching step.

The surface of the glass element base body may be treated by grinding and polishing, in particular before and/or after the etching. Grinding and polishing after the etching step have proven to be particularly advantageous, since they allow particularly planar surfaces to be achieved around the channel entrance and/or channel exit as well.

In some exemplary processes, hemispherical impressions/recesses are introduced at the inner channel wall by the etching.

This takes place in particular when a slow etching process is carried out, in particular with an etching rate of less than 15 μm per hour.

It is thought that the hemispherical recesses are caused by structures which occur when the filamentary flaws are inserted. An exemplary etching medium is a liquid etching solution. In accordance with such embodiments, then, etching is carried out wet-chemically. This may likewise be favorable for removing glass constituents from the surface during etching.

Both acidic and alkaline solutions may be used as etching solutions. Exemplary suitable acidic etching media are HF, HCl, H₂SO₄, ammonium bifluoride, HNO₃ solutions or mixtures of these acids. Candidates as basic etching media are optionally KOH— or NaOH solutions. With acidic etching solutions, greater ablation rates can be typically achieved. Basic solutions, however, may be preferred, since in any case the aim is only for slow ablation.

Etching may optionally be carried out in a temperature range from 40° C. to 150° C., optionally from 500 to 120° C. Silicatic glasses of low alkali metal content are particularly suitable in general for the structuring of the invention. If alkali metal contents are too high, they hinder etching. According to some embodiments, therefore, the glass of the glass element is a silicate glass having an alkali metal oxides content of less than 17 percent by weight.

The glass element with the micro-channels, as described, may be used advantageously for investigating biological material, more particularly human or animal or plant cells and/or cell constituents, more particularly proteins and/or DNA.

Alternatively to the spatially resolved diagnosis, the glass element, by virtue of its large number of blind holes, can be used for single molecule diagnosis or single cell diagnosis. In this case, a sample, more particularly a liquid sample, is introduced into the blind holes, by sweeping via a squeegee process or via the processes identified above, for example. As a result, depending on the choice of concentration, no molecule or cell for investigation, or only a few such molecules or cells, or exactly one individual such molecule or cell, enters a blind hole. At this point, in a targeted way, the molecules and/or cells for investigation in the individual blind holes can be multiplied, for subsequent implementation of analysis, more particularly of fluorescence analysis.

In some embodiments, in combination with a microfluidic cell which is able to address the individual blind holes or groups of blind holes in a targeted manner, specific tests are carried out for each of these blind holes or the blind hole group. This is of advantage particularly for medical diagnosis, to determine the heterogeneity of a sample or to perform numerous different tests in a small space (high-plex diagnostics).

The invention is elucidated in more detail below with reference to the figures. The figures and the associated observations represent examples; the invention is not restricted to these examples. In the figures, identical reference symbols each denote identical or corresponding elements. Reference symbols shown in one figure may also be valid for other figures, despite not being represented therein. The figures show diagrammatic exemplary embodiments; the proportions do not necessarily match the proportions or dimensions of the subjects of the invention. In the figures,

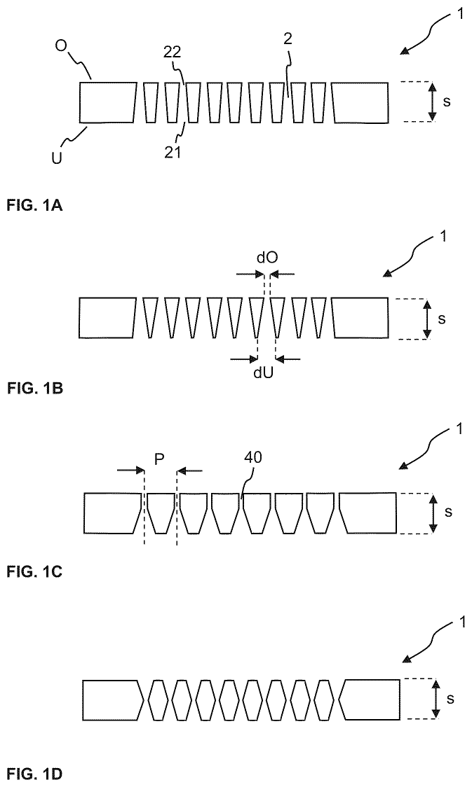

FIG. 1A shows one exemplary embodiment of a glass element 1 having a multiplicity of micro-channels 2. In this exemplary embodiment, the micro-channels connect the top side O of the glass element to its bottom side U and emerge through the latter. As described above, the bottom side is generally that surface or side of the glass element which faces away from the sample or on which the analysis takes place. The bottom side is generally that surface or side of the glass element which is facing the sample and hence the biological material for investigation. In the region of the bottom side U is located, in the terminology of this description, the channel entrance 21, and in the region of the top side O the channel exit 22.

In this example of FIG. 1A, the micro-channels taper in the direction from the bottom side U to the top side O, in this example continuously. Generally, the at least regional widening is called funnel-shaped in this description. In the example shown, the tapering is continuous. The profile of the micro-channels 2 may also be called frustoconical here. The region of the tapering extends here over the entire thickness s of the glass element.

The thickness s of the glass element after processing thereof may as described be in particular from 0.1 mm to 3 mm. The glass element 1 shown has substantially plane-parallel surfaces O and U. The same is usually true of the side faces. The glass element 1 is optionally a slide.

In FIG. 1B, a similar exemplary embodiment is represented, in which the tapering of the micro-channels is more marked. As a result of the tapering in the direction of the top side O and/or of the at least regionally funnel-shaped embodiment of the micro-channels 2, in the sense of the invention the diameter dU of the channel entrance 21 at the bottom side U is greater than the diameter dO of the channel exit 22 at the top side O of the glass element 1.

FIG. 1C shows a further exemplary embodiment, in which the profile of the micro-channels is composed of a tapering, frustoconical region in the region of the bottom side U and a cylindrical region in the region of the top side O. The overall shape may likewise be referred to as funnel-shaped. As described above, the center point of the channel exits 22 determines the pitch P. The aim is for as low a pitch as possible, since the micro-channel density increases with lower pitch. The pitch may be, as described, from 3 to 70 μm. As shown in this example, the center point of the channel entrance 21 is located substantially over the center point of the channel exit 22. In other words, both center points lie substantially on an axis which passes perpendicularly through the surfaces O and U of the glass element.

FIG. 1D shows an exemplary embodiment in which micro-channels 2 taper both from the bottom side U and from the top side O in the direction of the bulk of the glass material. In the form shown illustratively here, this produces an hourglass or X shape. This shape is composed here in principle of two frustrums placed invertedly one on top of the other, making contact at their narrow sides.

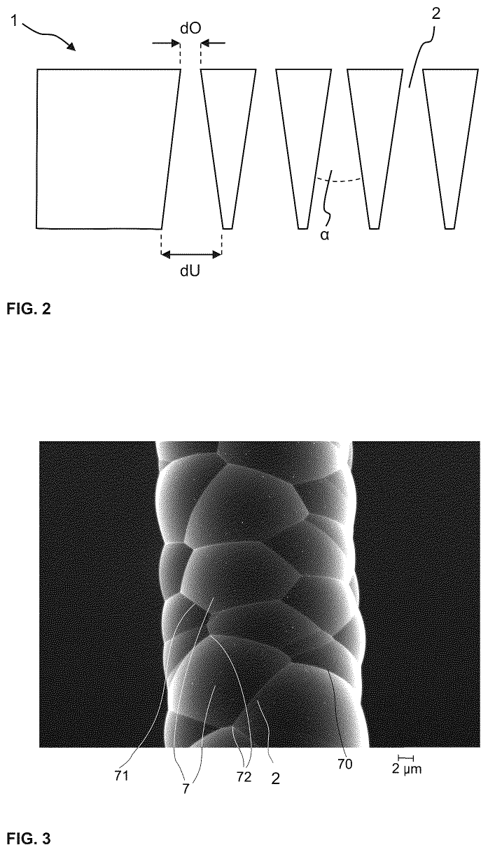

FIG. 2 shows a detail of a glass element 1 according to FIG. 1B. Drawn in here is the opening angle α, to be understood as the angle between the channel walls of a widened region of a micro-channel 2. The opening angle α is optionally from 0.10 to 30°, optionally from 2° to 18°, where the upper and lower limits in the intervals should of course be understood to be switchable.

As elucidated comprehensively above, the tapering micro-channels make it possible to resolve the conflict of objectives between on the one hand an extremely low pitch, to raise the resolution of the investigation, and also a high detection sensitivity and hence a large diameter of the channel entrance, and on the other hand the need to ensure the mechanical stability of the glass element for it to be rationally usable. In particular, in the region without tapering, there is sufficient material remaining to ensure the mechanical stability.

FIG. 3 shows the electron micrograph of the wall of a micro-channel 2 of a glass element 1 provided according to the invention, produced by the inventors, specifically in the detail of a region 40 of a micro-channel as shown in FIG. 1C. The cross-dimension, or diameter, of the channel 2 here is, as already stated, less than 200 micrometers, in this example about 14 micrometers. Without restriction to the examples described here, preference may be given to a diameter situated in the range from 5 micrometers to 200 micrometers.

Readily visible are the hemispherical or rounded, hood-shaped recesses 7 produced by the etching of the filamentary flaws introduced into the glass element by the ultrashort pulse laser beam. The depth of the hemispherical recesses 7 is typically less than 5 μm, for cross-dimensions of typically 5 to 20 μm.

As a result of the process of etching the filamentary flaws, the hemispherical recesses 7 border one another, with the abutting concave curvatures of the recesses 7 forming ridges 70. It is also apparent that the ridges 70, in a plan view of the recesses 7, form polygonal limiting lines 71 of the recesses 7. The mean number of the corners 72 of the limiting lines 71 of the recesses 7 here is optionally also less than eight, optionally less than seven. The latter feature comes about when the areas occupied by the majority of the hemispherical recesses are convex in the mathematical sense.

The ridges 70 of the channel 2 shown in FIG. 3 are very narrow; there are no recognizable regions in which the concave curvatures of the recesses 7 merge into one another via a convexly curved region at the ridge 70. According to some embodiments, therefore, the structure of the channels 2 may also be described such that the fractional area of regions convex in shape in a channel 2 is less than 5%, optionally less than 2%.

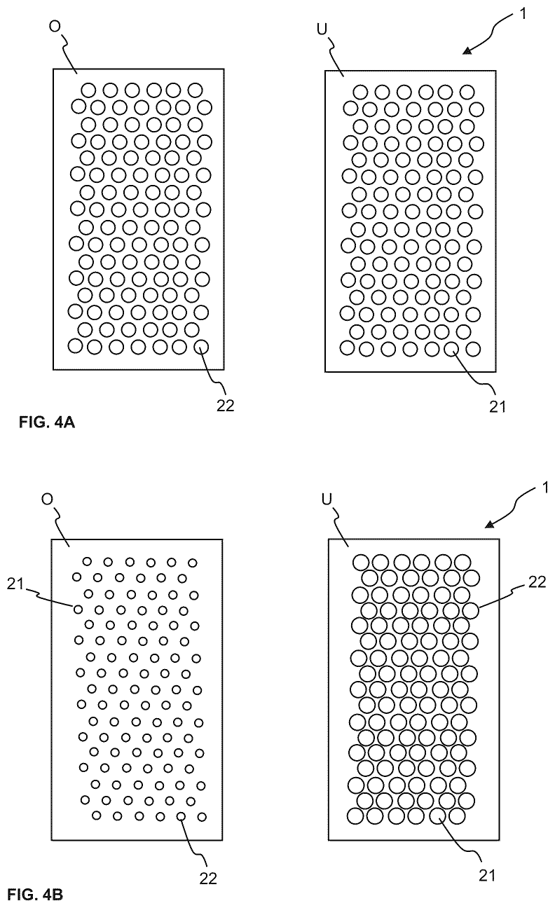

FIG. 4A represents, schematically, a plan view of the top side O and the bottom side U of a glass element 1. The diameters of channel entrances 21 and channel exits 22 are substantially the same. A pattern of this kind is to be expected, for example, in the case of an embodiment in accordance with FIG. 1D.

FIG. 4B shows a similar plan view, but the diameters of channel entrances 21 are smaller than those of channel exits 22. A pattern of this kind is present, for example, in the case of embodiments in accordance with FIGS. 1A to 1C.

As shown in FIGS. 4A to 4B, the channel entrances and exits form a matrix. Because the position of a channel entrance is assigned to a position of a channel exit, it is possible, with knowledge of the position of the channel exit, to determine with spatial resolution an event, for example the presence of DNA or particular biological material, on the side of the channel entrance and hence on the sample side.

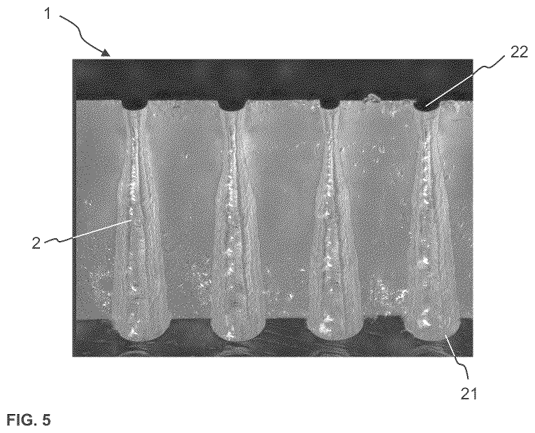

FIG. 5 depicts the photograph, taken through an optical microscope, of the section through a glass element 1 produced by the inventors and corresponding substantially to the embodiment of FIG. 1C. A region with funnel-shaped widening is adjoined by a cylindrical region of the micro-channel 2. The diameter of the channel entrance 21 at the bottom side of the glass element 1 is greater than the diameter of the channel entrance 22 at the top side. The glass used in the present exemplary embodiment was Schott D263. The invention is not restricted to particular varieties of glass. Other exemplary embodiments were produced with borosilicate glasses and/or alkali-free glasses. In particular, borosilicate glasses are frequently employed in the laboratory sector because of their chemical resistance qualities. As already described, there is particular advantage in glasses which exhibit little intrinsic fluorescence in the visible region of the spectrum.

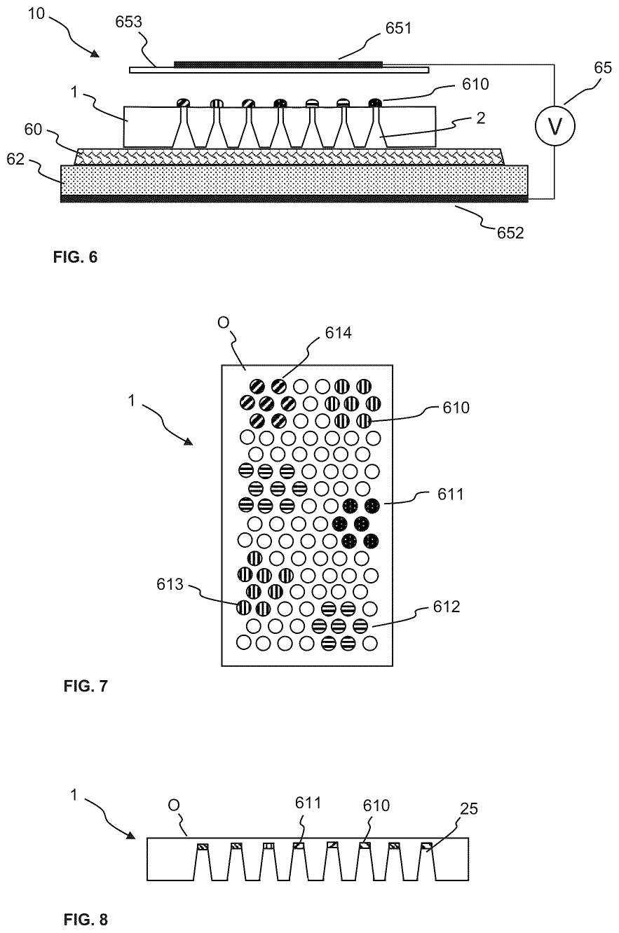

FIG. 6 shows an exemplary system 10 for the spatially resolved determination of biological material 60. The biological material for investigation is located here on a sample carrier 62. The glass element 1 having the described micro-channels 2 is located in contact with the biological material 60. It is of course also possible for the glass element 1 to be brought into contact with the biological material without being prepared on a carrier. For example, the glass element may be brought directly into contact with, for example, tissue or a cell culture.

Located in the micro-channels 2 there may be a gel, more particularly a gel as described. The gel is not represented in FIG. 6. On the top side of the glass element 1, at least one DNA- or RNA-sensitive dye 610 is attached in the region of the channel exit 22. If biological material 60 is passed, in particular with the aid of the gel, through the micro-channels 2, it arrives on the top side in contact with the dyes 610. The typical staining by the dye may then indicate the existence or, depending on the dye, the presence of a particular DNA strand. As described, the dyes are typically irradiated with excitational light and then the typical fluorescence of the dye is measured by an evaluation facility. For reasons of clarity, this figure does not show the evaluation facility. As likewise described, this facility may more particularly be an electronic camera.

There may advantageously be different dyes 610 attached at different channel exits 22. In the analysis of biological material, different DNAs and/or DNA strands may then be identified.

In the plan view, the system 10 shown produces a pattern from which conclusions may be drawn, with spatial resolution, regarding the presence of particular biological material, especially DNA. All statements made in relation to DNA are valid analogously in relation to RNA.

The transport of biological material 60 into/through the micro-channels 2 may be assisted by the presence of a transport facility 65. In the example in FIG. 6, this is a facility which in general utilizes an electrical potential for the transport of the biological material. The facility shown is a capacitor facility, in which a voltage source V is connected to the electrodes 651, 652. The electrodes in this example are disposed on the bottom side of the sample carrier 62 and on a non-conducting plate 653, more particularly a glass plate or plastic. The electrodes hence form a plate capacitor, with the biological material 62 and the glass element 1 being located between its plates. Application of voltage generates an electrical field by which the biological material and/or the medium surrounding it is pulled or pressured in the direction of an electrode 651.

The electrically non-conducting plate 653 may also serve as termination for the micro-channels 2 if it is brought into contact with the top side O of the glass element. This then produces, as it were, a blind hole, and the dye is not removed from the surface of the glass element by the advancing biological material.

The electrodes 651, 652 may be applied efficiently by coating, more particularly by sputtering.

Other configurations are likewise possible and encompassed by the invention. In particular, it is also possible to use pressure and/or negative pressure to transport the biological material, instead of electromotive force.

FIG. 7 shows the plan view of a glass element 1 provided on the top side O with sensitive dyes 610, 611, 612, 613, 614, more particularly in a system according to FIG. 6. The pattern represented shows, schematically, one possible outcome of investigation.

FIG. 8 shows once again, for clarity, the embodiment of a glass element having micro-channels 2 configured as a blind hole 25. Attached at the base of the blind holes are the aforesaid dyes 610, 611. The electrode 651 of an electrical transport facility 65 may easily be applied on the surface of the glass element. To enable the detection of dye fluorescence through the glass material located over the base of the blind hole, the electrode in this case is more particularly a transparent electrode, which may be represented for example as an ITO coating. Alternatively, it is also possible to configure the electrode as a grid and/or lattice, in particular with spaces over the base of the corresponding blind holes 25.

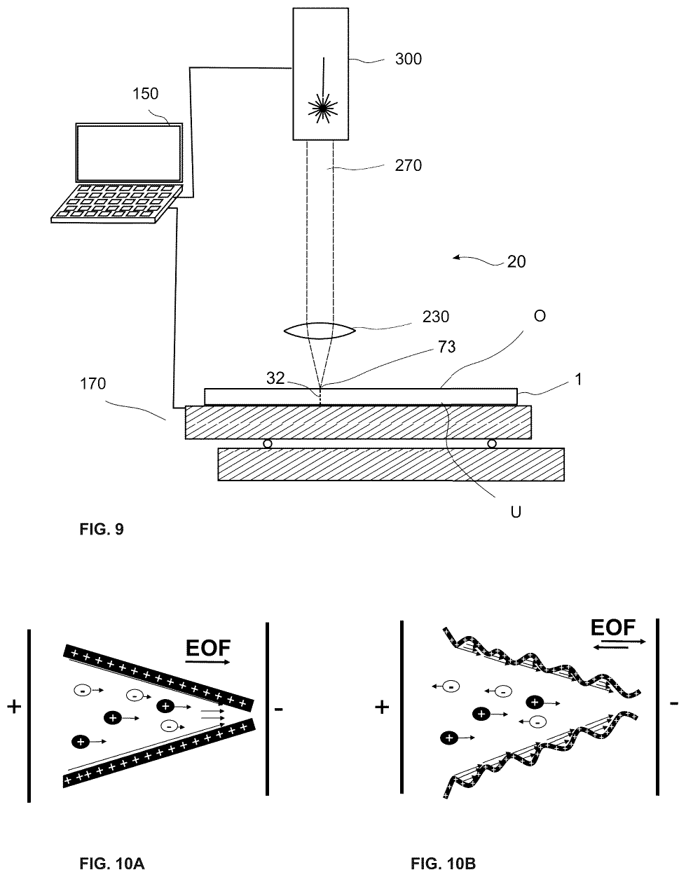

FIG. 9 shows an exemplary embodiment of a laser processing facility 20, with which filamentary flaws 32 can be inserted into a glass element base body 1, for subsequent insertion of micro-channels at the locations of the filamentary flaws 32 in an etching process. The apparatus 20 comprises an ultrashort pulse laser 300 with upstream focusing optical system 230 and a positioning facility 170. The positioning facility 170 can be used for lateral positioning of the impingement point 73 of the laser beam 270 of the ultrashort pulse laser 300 on the surface O of a platelike glass element 1 for processing. In the example represented, the positioning facility 170 comprises an x-y table, on which the glass element 1 lies on the bottom side U. However, it is also possible, alternatively or additionally, for the optical system to have a movable configuration, in order to move the laser beam 270, so that the impingement point 32 of the laser beam 270 can be moved, with the glass element 1 kept stationary.

FIG. 10A and FIG. 10B show schematically the effect of the structure of the micro-channel walls. Whereas a smooth channel wall is represented in FIG. 10A, FIG. 10B shows a structured channel wall having a roughness Ra. The channel walls have an opening angle. The structure may have the shape of the hemispherical recesses. EOF denotes the electro-osmotic flow. The electro-osmotic flow is, as described, a charge-induced movement of liquids along a polar surface in an electrical field. A greater roughness of the polar surface in accordance with FIG. 10B leads to a reduction in the velocity of the EOF and possible reversal of the direction of movement of the polar biological material in the glass element, in comparison to a surface with lower roughness in accordance with FIG. 10A.

The focusing optical system 230 then focuses the laser beam 270 on a focus which is elongated in beam direction, i.e. is elongated correspondingly transversely, more particularly perpendicularly, to the irradiated surface O. Such a focus may be generated, for example, using a conical lens (known as an axicon) or a lens with substantial spherical aberration and/or substantial chromatic aberration. Control of the positioning facility 170 and of the ultrashort pulse laser 300 is carried out optionally by a computing facility 150 with programming equipment. In this way, predetermined patterns of filamentary flaws 32 distributed laterally along the surface O can be generated, more particularly by the reading-in of position data, optionally from a file or over a network. The position of the focus allows the shape of the filamentary flaws 32 to be adjusted such as to result in local widening of the micro-channels 2 generated by the subsequent etching.

According to one exemplary embodiment, parameters used for the laser beam may be as follows: the wavelength of the laser beam is 1064 nm, typical of a YAG laser. A laser beam is generated that has a raw beam diameter of 12 mm and is then focused with an optical system in the form of a biconvex lens having a focal distance of 16 mm. The pulse duration of the ultrashort pulse laser is less than 20 ps, optionally about 10 ps. The pulses are delivered in bursts comprising 2 or more, optionally 4 or more, pulses. The burst frequency is 12-48 ns, optionally about 20 ns, the pulse energy is at least 200 microjoules, and hence the burst energy is at least 400 microjoules.

Subsequently, after the insertion of one or more particularly a multiplicity of filamentary flaws 32, the glass element 1 is removed and stored in an etching bath, where glass is removed along the filamentary flaws 32 in an etching process, so inserting a micro-channel 2 into the glass element 1 at each position of such a flaw 32.

Preference may be given to a basic etching bath having a pH>12, for example a KOH solution at >4 mol/l, optionally >5 mol/l, optionally >6 mol/l, but <30 mol/l. Etching according to one embodiment of the invention is carried out, independently of the etching medium used, at an etching bath temperature of >70° C., optionally >80° C., optionally >90° C.

The glass element and the system provided according to the invention have the advantage that they permit spatially resolved investigation of biological material. The glass element can be produced rationally and consists of materials which are commonplace in the laboratory environment. After use, the glass can be melted down again, meaning that the raw materials are recyclable.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A glass element for receiving and/or conveying biological material, the glass element comprising a multiplicity of micro-channels, the micro-channels tapering from a bottom side of the glass element in a direction of a top side of the glass element.

2. The glass element of claim 1, wherein the micro-channels each connect one surface of the glass element to an opposite surface of the glass element or end in a blind hole.

3. The glass element of claim 2, wherein at least one DNA-sensitive dye or RNA-sensitive dye is located in a region of channel exits or at a base of the blind holes.

4. The glass element of claim 2, wherein molecules for a polymerase chain reaction are located in a region of channel exits or at a base of the blind holes.

5. The glass element of claim 1, wherein the micro-channels are at least regionally funnel-shaped.

6. The glass element of claim 5, wherein an opening angle of a funnel-shaped region is from 0.10 to 30°.

7. The glass element of claim 5, wherein a funnel-shaped region is adjoined in a region of a channel entrance by a cylindrical or funnel-shaped region in a region of a channel exit or an end of a blind hole.

8. The glass element of claim 1, wherein a diameter of a channel entrance of one of the micro-channels at the bottom side is greater than a diameter of a channel exit or a channel end of the micro-channel in a region of the top side.

9. The glass element of claim 1, wherein the micro-channels at least in a region of a channel entrance or a channel exit or a channel end of the glass element have a diameter of 5 μm to 200 μm.

10. The glass element of claim 1, wherein a micro-channel density is at least 20 micro-channels/mm² up to at most 16,000 micro-channels/mm².

11. The glass element of claim 1, wherein a channel wall of the micro-channels has a structure with a multiplicity of rounded, hemispherical recesses having a depth of less than 5 μm.

12. A system for investigating biological material, comprising:
a glass element comprising a multiplicity of micro-channels, the micro-channels tapering from a bottom side of the glass element in a direction of a top side of the glass element, wherein the glass element in an operating state serves for receiving and/or conveying the biological material in the or through the micro-channels from the bottom side of the glass element into a region of the top side of the glass element; and
an evaluation facility with which in the operating state the biological material can be investigated.

13. The system of claim 12, wherein at least one DNA-sensitive dye or RNA-sensitive dye is located in a region of channel exits or at a base of blind holes, the evaluation facility being able to capture a color information of the at least one DNA-sensitive dye or RNA-sensitive dye.

14. The system of claim 12, further comprising a transport facility assigned to the glass element and in the operating state introduces the biological material for investigation into the micro-channels of the glass element and/or transports the biological material through the micro-channels.

15. The system of claim 14, wherein the transport facility comprises a negative pressure unit with which, in the operating state, negative pressure is applied to an exit side of the glass element, and/or a facility for generating a potential gradient over a thickness of the glass element so that biological material for investigation is introduced into and/or transported through the micro-channels by a flow or by an electrical field, and/or a squeegee unit with which biological material for investigation is introduced into blind holes by sweeping.

16. The system of claim 12, wherein biological material is multiplied in micro-channels and/or blind holes in the operating state.

17. The system of claim 12, wherein the evaluation facility comprises a closed or nanoporous membrane composed of a non-conductor or a semiconductor or a combination of non-conducting, conducting and/or semiconducting constituents on an exit side of the micro-channels.

18. A process for producing a glass element having a plurality of micro-channels which connect one surface of the glass element to an opposite surface of the glass element, the micro-channels tapering from a bottom side of the glass element in a direction of a top side of the glass element, the process comprising:
providing an ultrashort pulse laser;
providing a glass element base body;
directing a pulsed laser beam of the ultrashort pulse laser onto the glass element base body;
harmonizing a wavelength of the laser beam and a material of the glass element base body with one another such that the glass element base body is substantially transparent to the laser beam;
focusing the laser beam to a focus region which is elongated in a beam direction and which lies at least partly within the glass element base body, an intensity of the laser beam and an extent of the focus being of a magnitude such that the laser beam leaves a filamentary flaw in the glass element base body, wherein the focusing inserts a plurality of such filamentary flaws; and
etching open the filamentary flaws to give micro-channels.

19. The process of claim 18, wherein the filamentary flaws do not reach at least one surface of the glass element base body and an opening to this at least one surface is accomplished by the subsequent etching.

20. The process of claim 18, wherein the etching introduces hemispherical recesses at an inner channel wall of the micro-channels.
